(19) United States
(12) Patent Application Publication
(10) Pub. No.: US 2006/0141370 A1
(43) Pub. Date: Jun. 29, 2006

(54) Photomasks and methods of manufacturing the same

(76) Inventors: Suk-Pil Kim, Yongin-si (KR); I-Hun Song, Seongnam-si (KR); Won-Joo Kim, Suwon-si (KR); Seung-Hyuk Chang, Seongnam-si (KR); Hoon Kim, Siheung-si (KR)

Correspondence Address: HARNESS, DICKEY & PIERCE, P.L.C., P.O. BOX 8910, RESTON, VA, 20195, US

(21) Appl. No.: 11/319,725

(22) Filed: Dec. 29, 2005

(30) Foreign Application Priority Data
Dec. 29, 2004 (KR) 10-2004-0115074

(51) Int. Cl. G21G 5/00; A61N 5/00; G21K 5/00; G03F 1/00

(52) U.S. Cl. 430/5; 378/35; 250/492.2

(57) ABSTRACT

A photomask may include a reflection layer including a material capable of reflecting electromagnetic radiation, and at least one ion region. The ion region may be formed by implanting ions of an absorbent capable of absorbing electromagnetic radiation. The reflection layer may have a stack structure including a plurality of layers. The ions of the dopant may be implanted into at least one of the plurality of layers.

### PRIORITY STATEMENT

This application claims priority under 35 U.S.C. § 119 to Korean Patent Application No. 10-2004-0115074, filed on Dec. 29, 2004, in the Korean Intellectual Property Office (KIPO), the entire contents of which are incorporated herein by reference.

### BACKGROUND

1. Field of the Invention

Example embodiments of the present invention relate to photomasks and methods of manufacturing the same.

2. Description of the Conventional Art

Extreme ultraviolet (EUV) rays may be used in photolithographic manufacture of semiconductor devices. These semiconductor devices may have smaller pattern sizes, for example, pattern sizes of less than 100 nm.

In the EUV region, a reflection or reflective photomask may be used because of its reflective properties (e.g., its ability to reflect EUV light). A conventional EUV photomask may have an EUV absorbent pattern formed on a reflection mirror having a higher reflectance in the EUV region. The absorbent pattern may be created by coating an absorbent substance onto the surface of the mirror.

FIG. 1 is a cross-sectional view illustrating the structure of a conventional reflection photomask.

As shown, a conventional reflection photomask 1 may include a substrate 2 formed of silicon or glass, a reflective layer 3 formed on the substrate 2 and/or an absorbent pattern 4 formed on the reflection layer 3.

The reflection layer 3 may have a multilayer structure in which different kinds of films, such as, molybdenum-silicon (Mo/Si) and/or beryllium-silicon Be/Si may be stacked. The absorbent pattern 4 may be formed of, for example, a tantalum nitride TaN film that absorbs EUV rays.

When the reflection photomask 1 is exposed to EUV rays, the dimensions of the absorbent pattern 4 may be different from the dimensions of the pattern the mask forms on a silicon wafer 5. This difference is expressed in equations 1 and 2 below. Equation 1 shows the relationship of desired lengths between each of the patterns (e.g., designed space of critical dimension (CD)) of the absorbent pattern 4 and actual lengths between patterns formed (e.g., printed space CD) on the silicon wafer 5 corresponding to the absorbent pattern 4. Equation 2 shows the relationship between a desired length of a pattern (e.g., designed line CD) of the absorbent pattern 4 and a length of a pattern (e.g., printed line CD) formed on the silicon wafer 5 corresponding to the absorbent pattern 4.
Printed Space CD=Designed Space CD+2d×tan θ×M (Equation 1)
Printed Line CD=Designed Line CD+2d×tan θ×M (Equation 2)

In equations 1 and 2, d indicates the thickness of the absorbent pattern 4 protruding upward from the reflection layer 3, θ indicates the angle of incidence of the EUV rays based on the normal to the absorbent pattern 4, and M indicates a reduction factor.

In conventional photolithographic processes for semiconductor manufacture, the 2d×tanθ×M in Equations 1 and 2 may have a value based on the orientation of the side surface of absorbent pattern 4 and the value of θ, Equations 1 and 2 may have given values, since the side surface of the absorbent pattern 4 is vertical and θ may be given. Accordingly, the designed space CD may be different from the corresponding or actual printed space CD formed on the silicon wafer 5. In addition, the designed line CD of the elements of the absorbent pattern 4 may be different from the corresponding printed or actual line CD of the pattern formed on the silicon wafer 5. This may result in incorrect transfer of the designed shape of the absorbent pattern 4 to the silicon wafer 5.

### SUMMARY OF THE INVENTION

Example embodiments of the present invention provide photomasks (e.g., reflection photomasks), and methods for manufacturing the same, which may allow more accurate transfer of the designed shape of an absorbent pattern for absorbing electromagnetic radiation (e.g., EUV rays) to a silicon wafer in photolithography, and methods of manufacturing the absorbent pattern.

Photomasks according to one or more example embodiments of the present invention may be used more easily in higher resolution photolithography and/or photolithographic semiconductor manufacturing processes.

A photomask according to an example embodiment of the present invention may include a substrate, a reflection layer and at least one ion region. The reflection region may be formed on the substrate and may include a material capable of reflecting electromagnetic radiation. The at least one ion region may be formed within the reflection layer, and may be doped with a dopant capable of absorbing the electromagnetic radiation.

In another example embodiment of the present invention, a reflection layer may be formed on a substrate. The reflection layer may include a material capable of reflecting electromagnetic radiation. At least one ion region may be formed within the reflection layer, for example, by doping at least a portion of the reflection layer with a dopant capable of absorbing electromagnetic radiation.

In example embodiments of the present invention, the electromagnetic radiation may be extreme ultra violet radiation and/or extreme ultra violet rays.

In example embodiments of the present invention, the at least one ion region may be formed using ion implanting. The dopant may be oxygen.

In example embodiments of the present invention, the reflection layer may have a stack structure. The stack structure may include a plurality of layers, and the ion region may be formed by implanting ions of the dopant into at least one of the plurality of layers. In another example, the ions may be implanted into at least eight layers of the reflection layer. The stack structure may include a plurality of first layers and a plurality of second layers stacked alternately. The first layer may include a metallic material (e.g., Molybdenum), and the second layer include a semi-metallic material (e.g., Silicon).

In example embodiments of the present invention, the reflection layer may be formed by sputtering.

In example embodiments of the present invention, a photoresist layer may be formed on the reflection layer. The photoresist layer may be patterned to form a photoresist pattern, and, ions of the dopant may be implanted into at least a portion of the reflection layer based on the pattern of the photoresist. The photoresist pattern may be removed. The ions of the dopant may be implanted by irradiating a electron beam onto at least a portion of the reflection layer based on the pattern of the photoresist. The electron beam may be, for example, an oxygen ion beam, and the dopant may be, for example, oxygen. The photoresist pattern may be removed by, for example, etching. In another example, ions of the dopant may be implanted into at least a portion of the surface of the reflection layer.

### BRIEF DESCRIPTION OF THE DRAWINGS

Example embodiments of the present invention will become more apparent by describing in detail the example embodiments shown in the attached drawings in which:

FIG. 1 is a cross-sectional view illustrating the structure of a conventional reflection photomask;

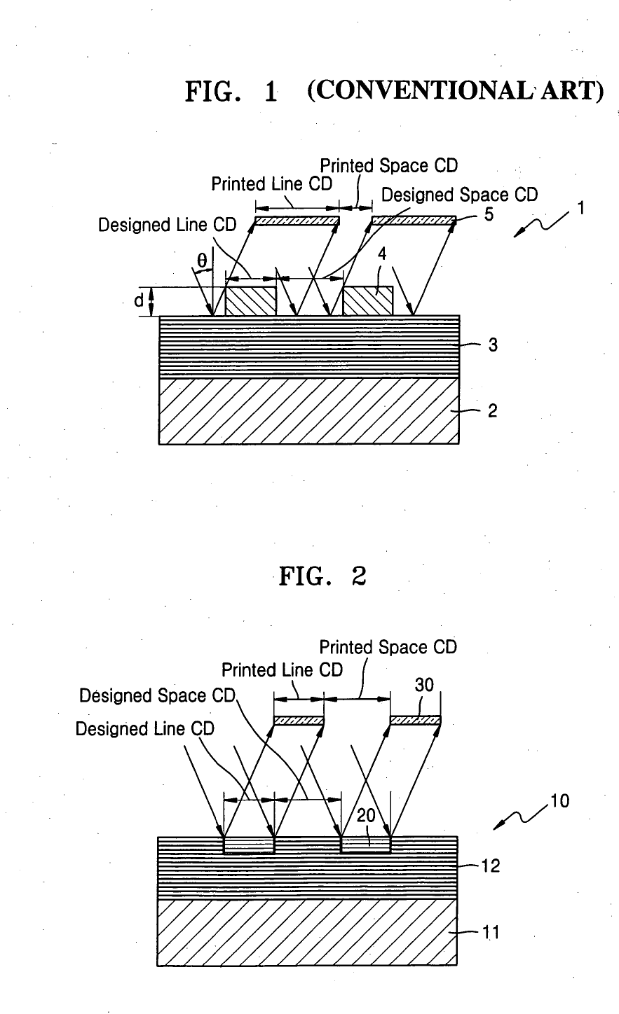

FIG. 2 is a cross-sectional view illustrating the structure of a photomask according to an example embodiment of the present invention;

FIGS. 3A through 3F are cross-sectional views illustrating a method of manufacturing a photomask according to an example embodiment of the present invention; and

FIG. 4 is a graph showing example results of an experiment on a photomask according to an example embodiment of the present invention.

### DETAILED DESCRIPTION OF EXAMPLE EMBODIMENTS OF THE PRESENT INVENTION

Example embodiments of the present invention will now be described more fully with reference to the example embodiments illustrated in the accompanying drawings. In the drawings, the thicknesses of layers and regions are exaggerated for clarity. Like reference numerals denote like elements throughout the drawings.

Various example embodiments of the present invention will now be described more fully with reference to the accompanying drawings in which some example embodiments of the invention are shown. In the drawings, the thicknesses of layers and regions are exaggerated for clarity.

Detailed illustrative embodiments of the present invention are disclosed herein. However, specific structural and functional details disclosed herein are merely representative for purposes of describing example embodiments of the present invention. This invention may, however, may be embodied in many alternate forms and should not be construed as limited to only the embodiments set forth herein.

Accordingly, while example embodiments of the invention are capable of various modifications and alternative forms, embodiments thereof are shown by way of example in the drawings and will herein be described in detail. It should be understood, however, that there is no intent to limit example embodiments of the invention to the particular forms disclosed, but on the contrary, example embodiments of the invention are to cover all modifications, equivalents, and alternatives falling within the scope of the invention. Like numbers refer to like elements throughout the description of the figures.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments of the present invention. As used herein, the term “and/or” includes any and all combinations of one or more of the associated listed items.

It will be understood that when an element or layer (e.g., a layer, a region and/or a substrate) is referred to as being “formed on” another element or layer, it can be directly or indirectly formed on the other element or layer. That is, for example, one or more intervening elements and/or layers may be present. In contrast, when an element or layer is referred to as being “directly formed on” to another element, there are no intervening elements or layers present. Other words used to describe the relationship between elements or layers should be interpreted in a like fashion (e.g., “between” versus “directly between”, “adjacent” versus “directly adjacent”, etc.).

The terminology used herein is for the purpose of describing particular embodiments only and is not intended to be limiting of example embodiments of the invention. As used herein, the singular forms “a”, “an” and “the” are intended to include the plural forms as well, unless the context clearly indicates otherwise. It will be further understood that the terms “comprises”, “comprising,”, “includes” and/or “including”, when used herein, specify the presence of stated features, integers, steps, operations, elements, and/or components, but do not preclude the presence or addition of one or more other features, integers, steps, operations, elements, components, and/or groups thereof.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

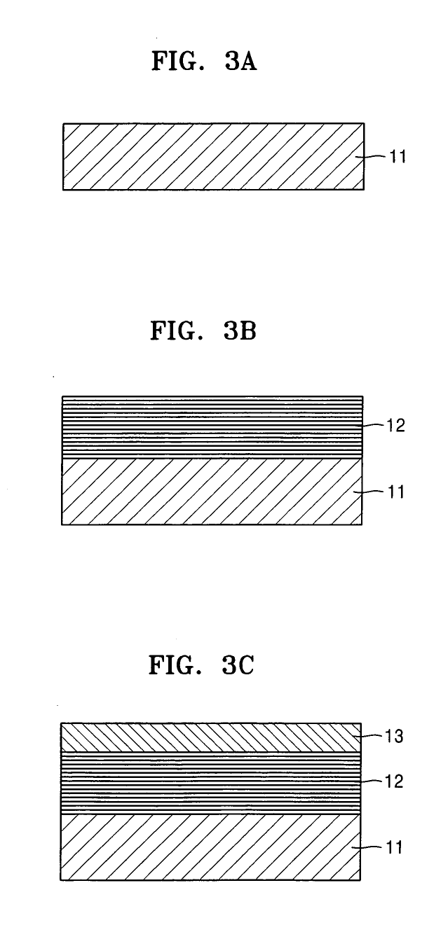

FIG. 2 is a cross-sectional view illustrating the structure of a photomask (e.g., a reflection or reflective photomask) according to an example embodiment of the present invention. As shown, a photomask (e.g., a reflection or reflective photomask) 10 may include a reflection layer 12 and/or an ion region or zone 20 formed within the reflection layer 12. The reflection layer 12 may be formed on a substrate 11. In example embodiments of the present invention, the substrate 11 may be formed of silicon, glass or any other suitable material.

The reflection layer 12 may have a single or a multilayer stack structure. For example, the reflection layer 12 may be a stack of layers, which may include a plurality of first films alternated with a plurality of second films. In example embodiments of the present invention, the first films may include molybdenum (Mo), molybdenum silicate, molybdenum carbonate, beryllium (Be), carbon (C), boron carbonate, or any suitable metallic film including elements and/or alloys having similar, or substantially similar, metallic and/or other properties.

In example embodiments of the present invention, the second films may include Silicon (Si), silicon dioxide (SiO₂) or any suitable semi-metallic film including elements and/or alloys having similar, or substantially similar, metallic and/or other properties.

In example embodiments of the present invention, an uppermost (e.g., the top) layer of the reflection layer 12 may be, for example, molybdenum, silicon or a combination thereof. An uppermost layer of silicon may allow a more stable natural oxide film (e.g., silicon dioxide SiO₂) to form on the silicon surface. A single film of molybdenum and/or silicon may have a thickness of a few nm (e.g., 1, 5, 10 nm, etc.), and any suitable number of layers may be stacked. For example, the reflection layer 12 may include 1 layer, 8 layers, 10 layers, 20 layers, 100 layers, etc.

According to example embodiments of the present invention, the photomask 10 may include an ion region 20. The ion zone or region 20 may be an EUV ray absorbing region having a pattern (e.g., desired or given pattern) of EUV ray absorbing material.

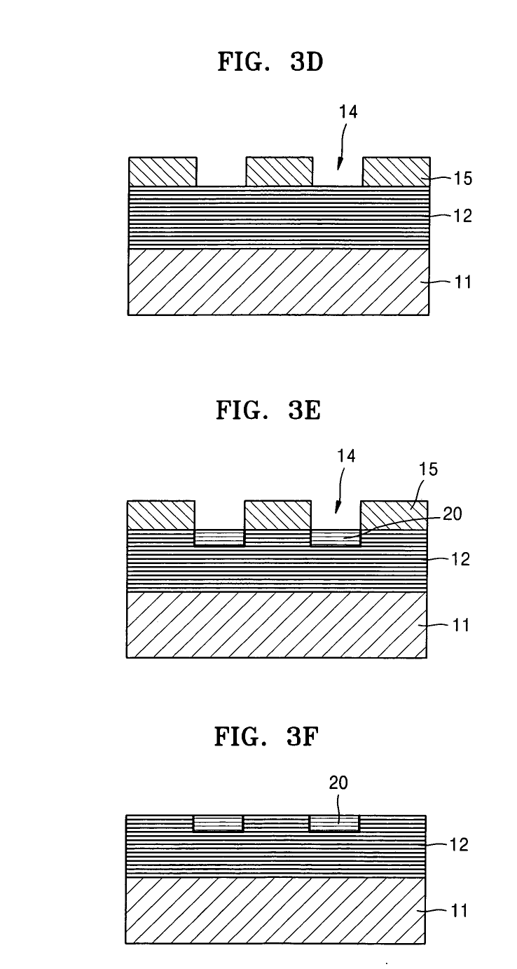

The ion region 20 may be formed by doping portions of the reflection layer 12 using an ion implantation method, which will be described with reference to FIGS. 3A through 3F.

In example embodiments of the present invention, the upper part of the reflection photomask 10 may be exposed to EUV rays, and the ion region 20 may be formed on portions of the surface of the reflection layer 12 exposed (e.g., directly exposed) to EUV rays. An example dopant for forming the ion region 20 may be oxygen, which may have higher EUV ray absorption. However, example embodiments of the present invention are not limited thereto, for example, any suitable material having similar, or substantially similar, absorption properties may be used.

If the ion region 20 is formed using oxygen, the dimensions of patterns formed on the wafer 30 may be identical, or substantially identical, to the dimensions of patterns formed on the ion region 20 when EUV rays are irradiated onto the reflection photomask 10. For example, the printed space CD and/or the printed line CD may be equal, or substantially equal, to the designed space CD and the designed line CD, respectively. This is shown in Equations 3 and 4.
printed space CD=designed space CD Equation 3
printed line CD=designed line CD Equation 4

Equations 3 and 4 show that the dimensions of the pattern formed by the mask 10 on the wafer 30 (e.g., printed space CD and printed line CD) may be equal, or substantially equal, to those of the ion region 20 (designed space CD and designed line CD), respectively.

In example embodiments of the present invention, the ion region 20 may be formed at and/or within an upper surface of the reflection layer 12, and the ion region 20 may not protrude upward from the reflection layer 12. As shown by contrasting Equations 1 and 2 with equations 3 and 4, the term ‘2d×tanθ×M’ of equations 1 and 2 is removed, for example, from equations 3 and 4 since there is no longer any portion corresponding to the thickness d of the conventional absorbent pattern 4. In example embodiments of the present invention, the designed shape of the ion region 20 may be more accurately (e.g., more correctly and/or correctly) transferred to the wafer 30.

FIGS. 3A through 3F are cross-sectional views illustrating a method of manufacturing a photomask (e.g., reflection photomask) according to an example embodiment of the present invention. In example embodiments of the present invention, oxygen may be used as an absorbent for EUV rays. However, any suitable element, gas and/or material with similar, or substantially similar, absorption properties may be used.

Referring to FIG. 3A, a substrate 11 may be prepared in any suitable manner. The substrate 11 may be prepared in any conventional well-known manner, and will not be described further herein for the sake of brevity.

As depicted in FIG. 3B, a reflection layer 12 including a plurality of layers of, for example, molybdenum and/or silicon may be formed on the substrate 11. In one example embodiment the layers of, for example, molybdenum and silicon may be alternately stacked. Radio frequency (RF) magnetron sputtering, ion beam sputtering or any other suitable formation method may be used to form the reflection layer 12. Sputtering conditions may be adjusted based on the apparatus used in forming the reflection layer 12.

As shown in FIG. 3C, a photoresist layer 13 may be formed on the reflection layer 12. The photoresist layer 13 may be formed in any conventional well-known manner, and will not be described further herein for the sake of brevity.

As depicted in FIG. 3D, a photoresist pattern 14 may be formed by exposing the photoresist layer 13 to energy such as an electron beam. The photoresist pattern 14 may be a mask exposing desired portions of the reflection layer 12 to form the ion region 20 in a desired pattern.

As depicted in FIG. 3E, the ion region 20 may be formed on the reflection layer 12. The ion region 20 may be formed by ion implanting, or any other suitable doping method. For example, after transforming oxygen into an ionized state, oxygen ions may be accelerated (e.g., to tens or hundreds of keV) using an ion implanting, or any other suitable apparatus. The oxygen ions may be irradiated onto the surface of the reflection layer 12 by a beam (e.g., an oxygen ion beam), and the ion region 20 may be formed by implanting the ions (e.g., oxygen ions) into the exposed upper surface portions of the reflection layer 12. For example, the oxygen ions may be implanted to 1, 8, 10, or any number of layers of the reflection layer 12.

As shown in FIG. 3F, the photoresist pattern 14 may be removed by etching or any other suitable removal process, and a photomask according to an example embodiment of the present invention may be manufactured.

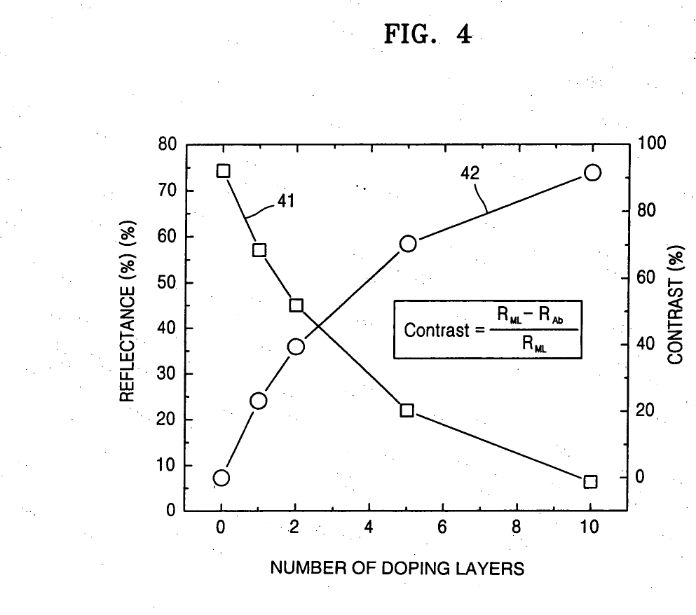

FIG. 4 is a graph showing example results of an experiment on a photomask according to one or more example embodiments of the present invention.

In the experiment providing the example results shown in FIG. 4, the reflection layer 12 was composed of a stack of 4.1 nm thick silicon films and 2.8 nm thick molybdenum films. An ion region 20 was formed by ion implanting stack layers of SiO₂ films each having a thickness of 5.2 nm and Molybdenum Oxide (MoO) films each having a thickness of 3.1 nm, respectively. In one example embodiment, layers of SiO₂ films and Molybdenum Oxide (MoO) films may be stacked alternately. The reflection layer 12 included a total of 40 layers with the ion region 20 being a portion of the reflection layer 12 comprising a total of 1 to 10 doping layers.

FIG. 4 shows the reflectance and contrast according to the number of doping layers in the ion region 20. As shown, the reflectance is the ratio between the intensities of the incident and reflected EUV rays of the ion region 20. The contrast is calculated according to Equation 5 shown below.
Contrast=(R_{ML}−R_{Ab})/R_{ML} Equation 5

In equation 5, R_{ML} represents the reflectance (e.g., in terms of a percentage) of the reflection layer 12, and R_{Ab} represents the reflectance (e.g., in terms of a percentage) of the ion region 20. Reference numeral 41 represents the variation of reflectance relative to the number of implanted layers of the ion region 20, and reference numeral 42 indicates the variation of contrast relative to the number of implanted layers of the ion region 20.

As shown in the graph, the reflectance of the ion region 20 may be approximately 58% and/or the contrast may be approximately 25% when one layer is implanted to form the ion region 20. The reflectance of the ion region 20 may be approximately 6% and/or the contrast may be approximately 90% when ten layers are implanted to form the ion region 20.

In one or more example embodiments of the present invention, the number of layers implanted to form the ion region 20 may be increased, the reflectance of the ion region 20 may decrease, the contrast may increase and/or the reflection and absorbing region of EUV rays may be more clearly distinguished.

Photomasks and the methods of manufacturing the same according to one or more example embodiments of the present invention may allow a designed pattern shape to be more accurately, more correctly and/or correctly transferred to the surface of a wafer (e.g., a silicon wafer) in photolithographic semiconductor manufacturing processes, for example, by forming an ion zone on a reflection layer.

Example embodiments of the present invention may use a reflection photomask, which may have increased contrast with an increase in implanted layers forming the ion zone. This may provide an improved photolithographic mask.

In example embodiments of the present invention, an absorbing zone for absorbing EUV rays may be formed without deposition and/or etching processes, which may simplify the manufacturing process.

Example embodiments of the present invention are described in conjunction with EUVL radiation. In example embodiments, EUVL radiation may be defined as radiation on the order of 1 to 30 petahertz (PHz), have a wavelength on the order of 10-100 nm, and/or have an energy of the order 12.4-124 eV. In other example embodiments, soft X-ray radiation may be used. In example embodiments, soft X-ray radiation may be defined as radiation on the order of 30 petahertz (PHz) to 3 exahertz (EHz), have a wavelength on the order of 100 pm to 10 nm, and/or have an energy of the order 124 eVto 12.4 keV. In still other example embodiments, any type of electromagnetic radiation may be used.

While example embodiments of the present invention have been described with reference to example embodiments shown in the drawings, it will be understood by those of ordinary skill in the art that various changes in form and details may be made therein without departing from the spirit and scope of the present invention.

What is claimed is:

1. A photomask comprising:
a substrate;
a reflection layer formed on the substrate, and including a material capable of reflecting electromagnetic radiation; and
at least one ion region formed within the reflection layer capable of absorbing the electromagnetic radiation.

2. The photomask of claim 1, wherein the electromagnetic radiation is extreme ultra-violet radiation.

3. The photomask of claim 1, wherein the at least one ion region is formed using ion implanting.

4. The photomask of claim 1, wherein the ion region includes a dopant capable of absorbing electromagnetic radiation.

5. The photomask of claim 4, wherein the dopant is oxygen.

6. The photomask of claim 1, wherein the reflection layer has a multilayer stack structure.

7. The photomask of claim 6, wherein the stack structure includes a plurality of layers and the ion region is formed by implanting ions of a dopant into at least one of the plurality of layers.

8. The photomask of claim 7, wherein the ions are implanted into at least eight layers of the reflection layer.

9. The photomask of claim 6, wherein the stack structure includes a plurality of first layers and a plurality of second layers stacked alternately.

10. The photomask of claim 9, wherein the first layer includes a metallic material and the second layer includes a semi-metallic material.

11. The photomask of claim 9, wherein the first layer includes molybdenum (Mo) and the second layer includes silicon (Si).

12. A method of manufacturing a photomask, the method comprising:
forming a reflection layer on a substrate, the reflection layer including a material capable of reflecting electromagnetic radiation; and
forming at least one ion region within at least a portion of the reflection layer, the ion region being capable of absorbing the electromagnetic radiation.

13. The method of claim 12, wherein the electromagnetic radiation is extreme ultra-violet radiation.

14. The method of claim 12, wherein the reflection layer is formed by sputtering.

15. The method of claim 12, wherein forming the ion region includes,
forming a photoresist layer on the reflection layer,
patterning the photoresist layer to form a photoresist pattern,
implanting ions of a dopant into at least a portion of the reflection layer based on the pattern of the photoresist, and
removing the photoresist pattern.

16. The method of claim 15, wherein the ions of the dopant are implanted by irradiating an electron beam onto at least a portion of the reflection layer based on the pattern of the photoresist.

17. The method of claim 16, wherein the electron beam is an oxygen ion beam.

18. The method of claim 15, wherein the photoresist pattern is removed by etching.

19. The method of claim 12, wherein forming the ion region includes,
implanting ions of a dopant into at least a portion of the surface of the reflection layer.

20. The method of claim 19, wherein the dopant is oxygen.

21. The method of claim 12, wherein the reflection layer has a multilayer stack structure including a plurality of layers, and the forming of the ion region includes implanting ions into at least one of the plurality of layers.

22. The method of claim 21, wherein ions are implanted into at least eight layers of ions.

23. A photomask produced using the method of claim 12.
